(19) United States
(12) Patent Application Publication
(10) Pub. No.: US 2015/0196691 A1
(43) Pub. Date: Jul. 16, 2015

(54) COATED STENT

(71) Applicant: AXETIS AG, Baar (CH)

(72) Inventors: Bruno Covelli, Suhr (CH); Nicolas Mathys, Baar (CH)

(73) Assignee: AXETIS AG, Baar (CH)

(21) Appl. No.: 14/420,009

(22) PCT Filed: Jul. 8, 2013
(86) PCT No.: PCT/EP2013/064341

§ 371 Date: Feb. 6, 2015

(30) Foreign Application Priority Data
Aug. 6, 2012 (CH) 01284/12

(51) Int. Cl. A61L 31/08; A61F 2/844; A61F 2/90; A61L 31/14

(52) U.S. Cl. CPC A61L 31/088; A61L 31/14; A61F 2/844; A61F 2/90; A61L 2420/02; A61L 2400/18; A61L 2400/16

(57) ABSTRACT

A coating (12) for a medical implant, particularly for a vascular stent (6). The coating comprises silicon dioxide and has a thickness of between 40 and 150 nm. Also, a method for producing such a coating, a coated medical implant, and a method for producing same.

### TECHNICAL FIELD

The present invention relates to a coating containing SiO₂, the coating being suitable for a medical implant, particularly a vascular stent, as well as a medical implant with a coating containing SiO₂, and a method for the production of the coating and the implant.

### PRIOR ART

Tubular support prostheses are well known in the prior art. They are often called “stents”.

For the purpose of keeping open vessels, such as blood vessels (e.g. arteriosclerosis), so-called stents are implanted into the occlusion-endangered vessels. This can be carried out by means of a catheter or by operative opening of the vessel, possibly by countersinking and implanting the stent. Stents are generally hose-like or tubular structures, for instance tissue tubes or tubular porous structures, which nestle to the inner wall of a vessel and keep open a free flow cross-section, through which the blood can flow freely in the blood vessel.

Further uses of stents are in billary tracts, in the trachea or in the esophagus. Thus stents are used, for example, in the treatment of carcinoma, for limiting the constrictions in respiratory tracts, billary tracts, the trachea or the esophagus after completed expansion.

Stents often consist of little tubes with a net-like wall, which have a small diameter and therefore can easily be brought to the place of action by means of a catheter, where they can be expanded to the necessary lumen and therefore to the necessary diameter for the support of the vessel by means of a balloon (balloon catheter) in the vessel by expansion of the net-like wall of the stent.

Balloon-expandable stents are typically produced from a formable metallic material, such as for example stainless steel or nickel-titanium alloys. Stents are usually formed by embossing selected structures out of tubes of the desired material. Examples of such machined processes are e.g. spark erosion (EDM—Electrical Discharge Machining), which is based on the erosion of metals by spark discharge, or laser beam treatment, in which a narrow light beam of high energy density is used in order to metalize or cut out selected sections of the metal tube.

These processes leave behind a thin heat-treated zone around the pattern cut in the tube, as well as a surface property which is rough and unsuitable for the implantation into live tissue. The surface property, i.e. the roughness or depth of roughness of stents on the outside and inside (Ra AD & ID) in the machined state usually is about 0.4 μm.

In order to smooth the stent surface, stents can be electropolished after the machined production. The principles of electropolishing as such, especially in connection with stainless steel alloys, are known from the prior art.

By coating the prosthesis, e.g. thrombocyte-aggregation and damages on the balloon catheter are avoided, and a minimizing of the surface roughness is achieved.

It is known to coat stents with plastics, such as for instance polytetrafluorethylene (PTFE; Teflon®).

From DE 102 30 720 A1, and DE 10 2005 024 913, vessel stents are known, which comprise a SiO₂-containing-, in other words a glass-like coating.

SiO₂-containing coatings, with or without additives, can basically be applied by known methods, such as e.g. by chemical vapor deposition.

Nevertheless, so far none of the developed methods for the production and coating of a medical implant has led to an optimal product, in which restenosis caused by intimahyperplasia is prevented.

Based on the increasing relevance of stents in the treatment of vessel diseases, an increased need exists for a constant improvement of the support function of the stents, while at the same time ensuring patient safety. Such implants especially should allow a non-problematic implantation in the body of a patient and at the same time decrease the intimahyperplasia.

### SUMMARY OF THE INVENTION

A too rough stent surface, as for example in a stent right after its machined production, can lead to serious complications, if such a stent is implanted in vivo. For example, the rough surface of the stent can offer the blood cells (e.g. thrombocytes, i.e. blood platelets) a surface, which promotes adhesion. Adhesion of such thrombocytes to the rough surface of a supporting prosthesis can trigger the sequence of steps, which is known as the coagulation cascade, which in severe cases can lead to the formation of a blood clot in and/or around the implanted prosthesis. If such a blood clot remains in this position, it can happen that the vessel closure, which actually shall be prevented by the vessel prosthesis, is caused again. If the blood clot detaches from the stent and wanders into the arterial or venous vessel system, it can possibly settle at a distant place in the body, can prevent the blood flow there and lead to an infarct or stroke.

Another negative effect of a rough surface of a vessel implant is the formation of undesired micro turbulences in the blood flow at this surface. The blood flow is diverted at smallest convexities. This deviation leads to micro-turbulences. Cell components can be caught in these turbulences and can also trigger the above mentioned coagulation cascade, with the according disadvantages and dangers for the patient.

This problem of providing an improved medical implant, which overcomes the above mentioned disadvantages, is solved by a coating according to independent claim 1, or a coating process according to claim 4, respectively, and a medical implant according to independent claim 7, or a process for the production of such a coated medical implant according to independent claim 13, respectively.

Accordingly, the invention is directed towards an improved medical implant and a process for the production of such an implant, wherein the implant comprises a coating containing silicon dioxide. Preferably the coating, besides incompletely oxidized reactant material, essentially comprises silicon dioxide. Preferably the medical implant is a vascular stent, for example for blood vessels, biliary tracts, esophagus' or tracheae. For example, EP 1 752 113 A1 discloses a vascular stent, which is suitable for the coating according to the invention, or as a support for an implant according to the invention, respectively.

An object of the present invention on the one hand is a coating comprising silicon dioxide for a medical implant, particularly a tubular supporting prosthesis. The tubular supporting prosthesis for example can be a vascular stent, such as e.g. a venous stent or an arterial stent, wherein the arterial stent can be implanted in the coronary artery or in the aorta. The stent can preferably comprise one or several artificial valves, and/or valves produced by tissue-engineering, e.g. an aortic valve.

Previously known stents (e.g. coated with PTFE or Teflon) have the problem that due to their specific surface and their lattice texture they often are overgrown or intermingled by autologous cells, which long term can lead to repeated occlusion of the vessel secured by a stent (restenosis). Here it is difficult to find the desired compromise between keeping open the vessel and harmonically integrating the stent in the organism. Also, conventional stent coatings are not always flexible enough to participate in the movements of the stent during implant and expansion, which can lead to damages in the coating. It has also been shown that between the substances of the stent and the blood or other tissue an electrochemical potential, or a voltage, respectively, can develop, wherein such potentials can change to the worse the properties of the blood components in the boundary layer and thereby lead to uncontrolled deposits such as plaques etc. These problems can partially be found also in other medical implants with similar requirements. The thickness of the coating lies about in the same range as the maximum tolerance for the surface roughness in the prosthesis.

Thereby the coating reflects the surface properties of the prosthesis, including the unevenness of the surface within the selected tolerances of roughness of the underlying prosthesis substrate.

Preferably, the thickness of the coating according to the present invention is 40-150 nm.

According to a preferred embodiment the thickness of the coating is in the range of 60-120 nm, preferably 80-100 nm, more preferably in the range of about 80 nm. The thickness is therefore preferably selected just in a way that a continuous layer results, which does not tear during movement or expansion of the implant, and preferably remains elastic at least in the area of use.

For the selection of the coating thickness, among others, the requirement is significant that during the expansion of the implant in the body the coating is not damaged and no additional pores are created.

The coating can be applied in one single step, and thereby can form a single-layer coat, however, according to a preferred embodiment it can also comprise several successively applied layers. In multi-layer processes, the composition of each layer can be individually determined.

The silicon dioxide can be present in amorphous or crystalline or half-crystalline form in the coating.

The properties of the coating can be further modified by at least one additive comprised in the coating, wherein the additive can be selected from aluminum oxide, titanium oxide, calcium compounds, sodium oxide, germanium oxide, magnesium oxide, selenium oxide, and hydroxides, particularly hydroxides of the aforementioned metals. Aluminum oxide and titanium oxide are especially preferred additives. If an additive to the silicon dioxide is used, the fraction of the additive in the total amount of the coating can preferably be 0.5 to 50 weight-%.

In order to retain the desired surface properties over the entire surface of the medical implant, such as a vascular stent, it is preferred that the coating is essentially free of pores.

In specific embodiments, however, it can also be preferred that the coating comprises pores for a functionalization with further substances, which are applied to the coating after the actual coating step, and which are deposited in the pores. Accordingly, the coating according to the invention can comprise an additional, functionalization coat, possibly only partially or punctually. Such a coating can correspond to the medical aim of the medical implant and can comprise an influence of the growth of surrounding tissue, or killing of unwanted tissue, or the establishment of a relation between medical implant and tissue, etc.

The functionalization coat can for instance contain at least one medication and/or at least one cell toxin.

The coating according to the invention preferably comprises a maximal mean defect size of 0.5-2 μm, preferably of about 1 μm. Thus, any possible tears or other damages in the SiO₂-layer preferably have a smaller diameter than 1 μm, or, respectively, the mean value of all defects on the surface of the coating before and/or after the expansion is 0.5-2 μm, preferably about 1 μm.

For the coating, advantageously a device for plasma-enhanced chemical vapor deposition (PECVD) (e.g a PECVD-reactor) is used.

Sonnenfeld et al. (A. Sonnenfeld, A. Bieder, Ph. Rudolf von Rohr, Influence of the gas phase on the water vapor barrier properties of SiOx films deposited from RF and dual mode plasmas, Plasma Processes and Polymers 2006, 3, 606-17) and Körner et al. (L. Körner, A. Sonnenfeld, Ph. Rudolf von Rohr, Silicon Oxide Diffusion Barrier Coatings on Polypropylene, Thin Solid Films 2010, 518(17), 4840-6) describe a possible plasma coating device and a possible coating process.

Plasma polymerisation is a special plasma-activated variant of the chemical vapour deposition. During plasma polymerisation, first of all, vaporous organic precursor compositions are activated in the process chamber by a plasma. By the activation, free charge carriers (ions and electrons) are created and first coating elements are already formed in the gas phase in the form of precursor fragments and/or clusters or chains of these fragments. The following condensation of these coating elements on the surface of the substrate, here the stent surface, brings about the polymerisation and thereby the formation of a closed layer, under the influence of substrate temperature, electron- and ion bombardment.

Such a process preferably comprises the following features:

A flow of process gas, comprising at least one gas (e.g. argon, Ar) and/or a gaseous oxidizing agent (e.g. CO₂, N₂O, O₃ or O₂) and a flow of carrier gas, comprising at least one precursor, are guided into a treatment zone, in which at least one substrate is present. The volume of the treatment zone is enclosed by the process chamber which can be evacuated.

Preferably, the flow of process gas and the flow of carrier gas each have at least one separate inlet port spaced apart from the other in the treatment zone. Advantageously, the process gas flow and the carrier gas flow each have several inlet ports. These can be realized by a hole or several holes in the wall of at least one e.g. ring-, rod-, string-like or otherwise formed hollow body (gas shower). The at least one gas shower is connected to the treatment zone via the aforementioned holes. Therein, the holes comprise characteristic widths in the range of 0.1-10 mm, preferably of 0.2-0.5 mm. In case of the coating of the stents, preferably ring-like gas showers are used, which are advantageously integrated in the vessel wall.

For the plasma activation, at least one preferably anisothermic, electric gas discharge is carried out in the process chamber. For this purpose, the production of an electric potential gradient (of a voltage) is necessary, with the help of at least one plasma source, by means of which the energy feed is carried out by radiofrequency- (RF-) or micro wave- (MW-) feeding. Typically, the voltage is applied over the distance between at least two electrodes (measuring electrode and counter electrode). Therein, the electrodes can be located inside and outside of the process chamber, i.e. at least one electrode outside and at least another inside the process chamber. At least one electrode can form a part of or the entire wall of the process chamber. Preferably (in the case of the stent), this is the measuring electrode.

Thus, several spaced-apart plasma zones can be achieved in the treatment zone, as well as one single connective plasma zone. Thus it is possible to either activate the process gas flow or the carrier gas flow, or both separately. Furthermore, the mixture of none, one or both already activated gas flows (process gas flow and carrier gas flow) can be activated in at least one plasma zone. The at least one plasma zone can fill out the entire treatment zone or it can make up a partial region of the treatment zone. Typically, the substrate is located downstream, in relation to the aforementioned inlet-ports of process gas flow and/or carrier gas flow. Therein, the substrate can be located inside or outside of the at least one plasma zone. Preferably the at least one substrate is supported by one of the aforementioned electrodes, or by a holding device supported by it. It is possible to make it dynamic, so that the at least one substrate can he freely moved in the treatment zone and thus can switch between direct plasma activation (substrate within a plasma zone) or remote plasma activation (in the after-glow) during the coating. Preferably, a heterogenous, chemical reaction of the coating elements takes place on the surface of the substrate. Preferably, exclusively a RF-plasma source is used for the deposition of the silicon-oxidic (SiO₂) layers on the stents (RF-mode). In the RF-mode, a holding device (in the form of a plate) with separate, electrically isolating holding elements lies on top of the counter electrode provided inside the process chamber.

Preferably, furthermore an active cooling of the counter electrode is used (e.g. by means of an integrated water heat exchanger), in order for the heat strain to be further reduced. A cooling temperature in the range of TE=15-45° C., preferably of 18° C.-25° C., and more preferably of about 20° C. has been shown to be advantageous.

During the production of the coating, besides the temperature of the counter electrode, the following parameters are important values for the achievement of a homogenous and smooth surface: wall temperature of the process chamber TPK (preferably 50° C.), pressure p, fed plasma power PRF, gas composition during the cleaning- and coating process (ratio of the gas volume flows [O₂]/[Argon], [O₂]/[HMDSO]), coating time t_{B}, as well as positioning of the probes in the reactor.

From case to case, the coating step can be preceded by a plasma-fine cleaning, wherein the concentration of the gaseous oxygen preferably is 100 sccm for 2×10 sec (seem: standard cubic centimeters per minute). The other parameters correspond to those of the coating step.

In a preferred method for the production of a coating according to the invention, O₂ and hexamethyldisiloxane (HMDSO or C₆H₁₈OSi₂) are used as reactants for the plasma polymerisation, wherein the oxygen is used as an activating gas and the hexamethyldisiloxane as a layer-former (precursor). Therein, a ratio of [O₂] to [HMDSO] (silicoorganic monomer) of in the range of 10:1 to 40:1 is especially advantageous, especially in the range of 10:1 to 20:1. According to an especially advantageous embodiment of the process for the production of the coating, a ratio of [O₂] to [HMDSO] of 14:1 to 18:1 is used, more preferably of about 15:1. According to an especially preferred production process, HMDSO is not completely oxidized. In other words, at least one part of the starting material is present in chain- or net-form in the final product. Preferably, only 80-95%, preferably about 90% of the starting material underwent a reaction, or only 80-95%, respectively, preferably about 90% of the starting material are present in the layer in chain- and/or net-form. This leads to the result that the resulting coating has optimal mechanical properties for the purpose of implanting, and cooperates in an especially advantageous way with the surface of the implant.

In an especially preferred embodiment a flow rate of O₂ of 60 sccm is used, at a flow rate of HMDSO of about 4 sccm, a preferred plasma power of 200 W, a preferred coating time of 2×6 sec, and a preferred reactor pressure of 0.14 mbar.

A great advantage of the medical implants according to the invention is to be seen in that the coating can be applied in an extremely thin manner, i.e. preferably in the nano-range, thus in the range of a couple of atomic layers. This allows to essentially adjust the end values during the production of the medical implant, without having to take into consideration possibly unforeseeable dimension changes of the coating. Furthermore, such a thin coating is less prone to break.

The invention is furthermore directed towards a medical implant, which comprises a support forming a basic structure and produced especially according to the above mentioned parameters, and a coating applied to at least parts of the support, the coating comprising or consisting of silicon dioxide. The coating is especially a coating according to the first aspect of the invention. Preferably, the medical implant is a vascular stent. The vascular stent can be determined for a blood vessel, a biliary tract, the esophagus or the trachea, wherein it can be used in various animal species, such as humans, pets, and farm animals.

The support is preferably formed of a difficult to degrade material, wherein “difficult to degrade” is to be understood as a property, in which the material does not show any visible signs of degradation for at least one year after implantation into a body. The support is preferably formed of materials usually used for medical implants, particularly comprising carbon, PTFE, Dacron, metal alloys, or PHA, wherein iron- or steel alloys, respectively, are especially preferred.

A further preferred material for the support is a metal having shape memory, particularly nickel-titanium alloys, which find use in stents due to their ability to change their form by themselves. However, also an aluminium alloy, magnesium alloy or an iron alloy can be used.

Furthermore, in a further aspect, the invention is directed towards a process for the production of a coated medical implant, particularly a medical implant according to the invention, which comprises at least the following steps:
providing a support forming a basic structure;
electropolishing the support;
applying a coating comprising silicon dioxide by means of a plasma coating process.

The support is, as mentioned above, preferably produced from a tubular metal blank of stainless steel, by cutting the blank in a laser cutting process. Therein, a stent structure is cut with the laser. The construction drawing of the stent is converted by a software into a format that is understandable by the CNC-controlled laser cutter, the so-called cut drawing (CNC: computerised numerical control). After inserting the tube, the following feeding is conducted preferably in a fully automated manner. The first stent of a production batch is controlled with respect to its even structure and cutting mistakes immediately after cutting.

The optical control is carried out under a microscope. Cutting mistakes are to be understood as contours contrary to the cut drawing. Furthermore, an exact measuring of the stent takes place by means of a profile projector or measuring microscope. If all parameters correspond to the specifications, the processing of the tube is continued.

The laser cutting process preferably comprises one or more of the following parameters:
continuous wave pulse transmission;
mean power of in the range of 5-9 W, at a power of at the most in the range of 80-100 W;
frequency of in the range of 5000-8000 revolutions/sec;
shutter speed in the range of 10-12 μs;
energy in the range of 0.8-1.2 mJ;
cutting speed in the range of 2-4 mm/sec;
positioning time of in the range of 5-10 mm/sec.

An especially preferred laser cutting process is characterized by one or more of the following parameters:
continuous wave pulse transmission;
mean power of 7.21 W, at a power of at the most 91.2 W;
frequency of 7000 revolutions/sec;
shutter speed of 11.3 μs;
energy of 1.03 mJ;
cutting speed of 2.76 mm/sec;
positioning time of 7.5 mm/sec.

After the laser cutting of the stents, they are preferably submitted to a subsequent etching process. A preferred etching solution comprises deionized water, nitric acid (HNO₃) and hydrofluoric acid (HF). An especially preferred composition comprises 75-80%, preferably 77.5% of deionized water, 18-19%, preferably 18.3% of nitric acid, and 4-4.5%, preferably 4.2% of hydrofluoric acid, tempered to 60-70° C., preferably 65.5° C.

After the laser cutting and a possible etching process, the stents are electropolished.

Typically, a product that is to be electropolished is immersed in an electrolyte, which contains an aqueous acidic solution. The product is formed to a positive electrode (anode), while a negative electrode (cathode) is placed close to the anode. The anode and cathode are then connected to a source of an electric potential difference, while the electrolyte closes the circuit between anode and cathode. After the flow moved through the electrolyte, the metal melts off the surface of the anode, i.e. off the surface of the medical implant to be polished, e.g. the tubular support prosthesis. Therein, projecting portions are melted generally faster than indentations, so that the surface is smoothened. The velocity of the discharge of material during electropolishing is primarily a function of the electrolyte and the flow density in the electrolyte fluid.

During the production process of tubular support prostheses, one attempts to maximize the efficiency. This is achieved during electropolishing after the machined production starting from the metal tube by an increase in velocity, for example by increasing the concentration of acid in the electrolyte bath, and/or by increasing the flow density. While such measures often are able to reduce the surface roughness to a satisfying degree, so that the aforementioned disadvantages concerning coagulation can be avoided, or at least are avoidable in vivo, the inventors have found out that an acceleration of the electropolishing process can also lead to very sharp edges of the sections cut out of the metal tube. The fast removal of material from the inner, outer and inner intersecting (transversal) areas can lead to the fact that the remaining portions accumulate at the edges, which can lead to sharp metallic edges at the places where the discharged areas intersect. Such sharp cutting points can interfere with the implantation process, during which the stent is spanned by means of a balloon catheter. For example, the balloon can be damaged by the sharp edges, which leads to a loss of pressure inside the balloon catheter. Thereby, the complete expansion of the stent, which is necessary so that the stent abuts optimally to the vessel, can be prevented. In such situations, the balloon catheter must be removed and the stent could get lost in the body and thus lead to life-threatening complications. Even in the case where the balloon itself is not damaged, and the stent is immobilized correctly at the right position, a sharp edged stent can still lead to severe complications. The sharp edges of the stent can be pressed against the inner wall of the vessel and gradually lead to irritations. Thus, inflammatory processes can be triggered at the site of the stent expansion, and in severe cases, a cicatrisation can lead to vascular constriction or stenosis.

In other words, typical production processes of tubular support prostheses by means of conventional machined processing of metallic tubular blanks, followed by electropolishing to improved smoothness of the implants, at the costs of sharp edges or, contrary thereto, rounded (previously sharp) edges at the cost of increased surface roughness. The inventors have found out, that faster or more aggressive electropolishing rather leads to smooth surfaces but sharp edges, while a slower or more mild form of electropolishing rather leads to rounded cutting edges but to more rough intermediate surfaces of the prostheses, wherein this is achieved for example by a process with the parameters as described below.

These correlations seem to mutually exclude each other and one supposes that a tubular support prosthesis necessarily comprises at least one disadvantage.

The inventors have now surprisingly found out that known processes for electropolishing can be carried out in a way that an advantage can be achieved, without having to give up of another advantage. Thereby, implants can be produced which avoid the undesired formation of thrombosis and simultaneously ensure a safe expansion by undamaged balloons, whereby irritations of the surrounding tissue can be avoided. In the prior art, it has so far not been possible to reach both goals simultaneously. In other words, known electropolishing processes can be conducted in a sufficiently fast and aggressive manner in order to achieve smooth surfaces, but not as fast and aggressive as to leave behind too sharp edges. The person skilled in the art therefore can adapt the parameters of the electropolishing process in an optimal way.

In the electropolishing process according to the invention, the stents are hung up on a rack of noble metal wires, which itself is connected to a polishing device. The rack can for instance be loaded on four wires with up to 20 stents each. Subsequently, the loaded rack is immersed in the electropolishing bath. In the electropolishing bath, the electric current, the temperature and the polishing time, as well as the charge quantity are regulated. A planetary gear on the polishing rack guarantees an even movement of the wires with the stents. The polishing fluid is a special mix of different acids. The quality of the polishing fluid is monitored by an aerometer. By means of a fine scale, each separate stent is weighed, and possibly re-polished, in order to guarantee the normal weight by +/−0.2 mg.

The electropolishing of the support takes place in an electrolyte bath. This advantageously contains at least phosphoric acid, sulphuric acid and distilled water. The electropolishing is carried out at a temperature of 70-74 degrees Celsius, preferably at a temperature of 70.3-73.5 degrees Celsius.

Therein it is preferred if the rotational velocity is adjusted to 2-6 mm/sec, preferably about 4 mm/sec.

The maximum applied voltage lies in the range of 3-4 V, and is about 3.5 V, preferably at the most 3.11 V. Therein, preferably a current of at the most in the range of 3-7 A, preferably of at the most 5 A flows. According to an especially preferred embodiment the support is electropolished for 300-500 sec, preferably for 440-470 sec, particularly preferably for 455 sec.

The maximum mean defect size at the support surface (i.e. in the present case after the electropolishing) advantageously is 0.5-2 μm, preferably about 1 μm, i.e. the support should not have any damage with a diameter larger than 0.5-2 μm, preferably no damage with a diameter larger than about 1 μm.

The still uncoated support (i.e. in the present case after the electropolishing) advantageously has a mean surface roughness Rₐ of at the most about 30 nm, preferably of at the most 20 nm. The mean roughness Rₐ defines the mean distance of a measuring point on the surface to a mean centerline. The centerline intersects the real profile within the reference distance such that the sum of the profile deviations (with respect to the centerline) becomes minimal. The mean roughness Rₐ therefore corresponds to the arithmetic mean of the deviation from a centerline. The roughness on the surface is standardized by ISO 25178. By means of optical measuring devices the value of roughness can be measured in terms of surface area (e.g. by means of the optical microscope VHX 100 of Keyence, with software-supported 3D-surface analytics and a resolution of 54 MPixel in combination with an up to 2500× optical magnifying lens of Zeiss. The software allows a virtual section through the surface and calculates the mean roughness depth for this measuring area).

Because thrombocytes, i.e. blood platelets, usually vary in their size between 2-4 μm, it can be guaranteed, by complying with the maximum surface roughness, that no thrombocytes get caught on the implant, which in turn decreases the risk of undesired complications due to prosthesis-induced coagulation.

The definition of an area of the surface roughness is furthermore important because the coating applied to the surface should remain dynamic, or flexible, respectively, i.e. not rigid, but at the same time should also not slide off the support surface. The quality of the surface to be coated therefore plays an important role in the layer formation.

Everything said with respect to the coating or the medical implant shall also extend analogously to the method according to the invention and vice versa, so that reference is made in an alternating manner.

In order to obtain the pores desired in specific embodiments in order to receive functionalization agents, it is furthermore preferred that the method also comprises the step of the production of pores in the coating by means of neutron bombardment. For this purpose, neutron sources such as for example particle accelerators can be used. A further possibility for the production of functional pores lies in the production of pores by means of laser light.

The present invention provides a coating for medical implants, particularly vascular stents, which essentially prevents, due to its inert, glass-like surface with silicon dioxide, an ingrowth of cells of the body, or an attachment of such cells, respectively, which due to its hardness counteracts a damage when introducing the implant into the body, and thereby simplifies the handling, which allows a more simple design of the implant due to the thinness of the coating, and leads to a reduced friction due to lower roughness values and therefore a smaller burden for blood components and to reduced coagulation, and when using such a coating, there is no degradation of the coating even after longer presence in the body.

Further embodiments are described in the dependent claims.

### BRIEF DESCRIPTION OF THE DRAWINGS

Preferred embodiments of the invention are described in the following with reference to the drawings, which are only for the purpose of illustration and not for limitation. In the drawings,

FIG. 1 shows an exemplary embodiment of an electropolished stent according to the invention, prior to being coated;

FIG. 2 shows a three-dimensional microscopic view of an excerpt of the surface of a stent of FIG. 1 as a basis for the measurement of the surface roughness, visualized in a ConScan white confocal microscope (CSM Instruments), in white light of 2 μm diameter; a scan-size of 0.25 mm×0.25 mm and a resolution of 1000 pixel/mm.

FIG. 3 a three-dimensional microscopic view of an excerpt of a coated stent according to the invention, visualized in a Olympus SZX12 light microscope, photographed by a Olympus ColorView Illu camera.

FIG. 4 a three-dimensional microscopic view of an excerpt of the coated stent of FIG. 3 according to the invention, visualized in a Zeiss Auriga scanning electron microscope, in a 400-fold magnification.

FIG. 5 a three-dimensional view of an excerpt of a coated stent according to the invention, visualized in a scanning electron microscope, in a 103-fold magnification; definition of the analysed stent sections after the dilatation;

FIG. 6 a three-dimensional microscopic view of an excerpt of a stent coated with SiO₂ according to the invention without a platinum coat, visualized in a scanning electron microscope, in a 50,000 fold magnification;

FIG. 7 a schematic presentation of the reactor for coating;

FIG. 8 a schematic presentation of the substrate holder in the reactor.

### DESCRIPTION OF PREFERRED EMBODIMENTS

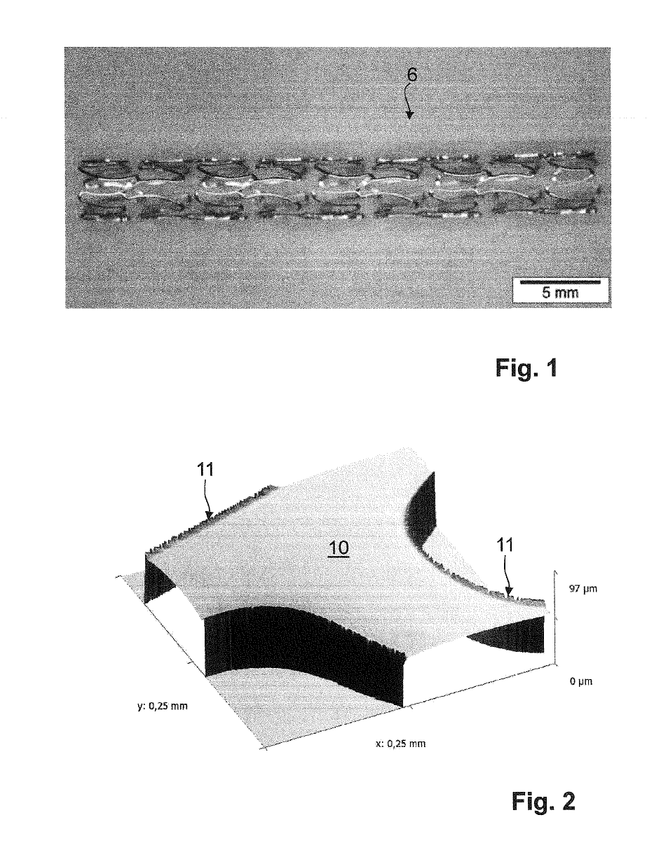

In FIG. 1, an uncoated support or a vascular stent 6, respectively, is shown, as it results from electropolishing. The mesh of the depicted stent 6 has several support rings 8 connected to each other at different places, wherein the support rings 8 each are formed by a filament wound to several arcs of curvature in a meander-like manner. Thereby, at least one arc of curvature of a first support ring and an are of curvature of a neighboring second support ring laterally overlap, wherein the connecting point is formed in the overlap area.

It can be seen on the vascular stent 6 shown in FIG. 2 that after electropolishing, the surface 10 seems very smooth. Some of the edges 11 of the still uncoated stent however, still are sharp.

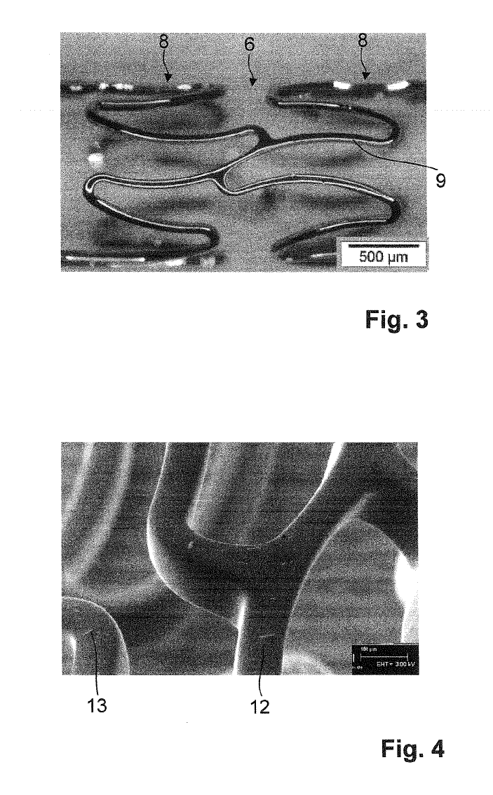

The excerpt of the coated vascular stent of FIG. 3 shown in FIG. 4 shows a continuous coating 12 with only minor damages 13. The morphology of the SiO₂-coeating 12 is strongly determined by the roughness of the underlying substrate surface 10. If this is rough, there will also be non-homogenous layer structures. For evaluating the quality of the coatings and for the differentiation between fine differences in the dilatation behavior, for example the electrochemical impendance spectroscopy (EIS) can be used.

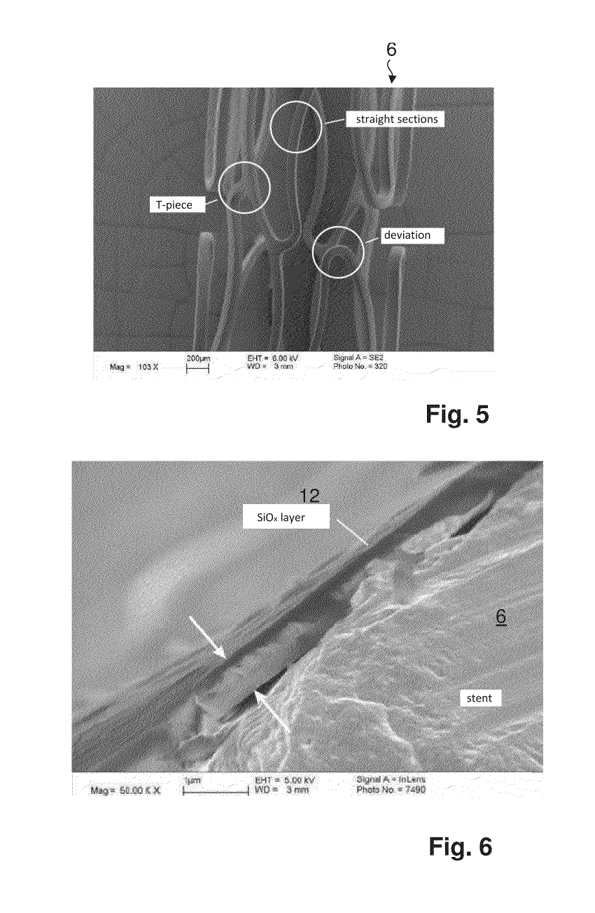

In the stents which form the basis for the present invention, the dilatation was examined in that the stents were expanded to different degrees, i.e. by 0%, 25%, 50%, 75% and 100% by a balloon catheter, and analysed in a scanning electron microscope (Zeiss, Gemini 1530 FE). The deformation of the stent according to the invention occurs only at the connecting areas (T-parts) and at the “deflecting areas” due to its special design. Accordingly, the damages 13 of the coating 12 primarily also occur at these strongly stressed areas (see FIG. 5).

In FIG. 6, an excerpt of a stent surface 10 is shown close to the section area with view of the section of the layer. The layer density equals about 600-800 nm here. Such large layer thicknesses have shown to be too large in order to ensure a sufficient elasticity of the layer-stent-conjunction. Thinner layers of about 200 nm showed significantly better deformation- and adhesion properties during a maximum expansion of the stent, compared to thicker layers of about 300-400 nm.

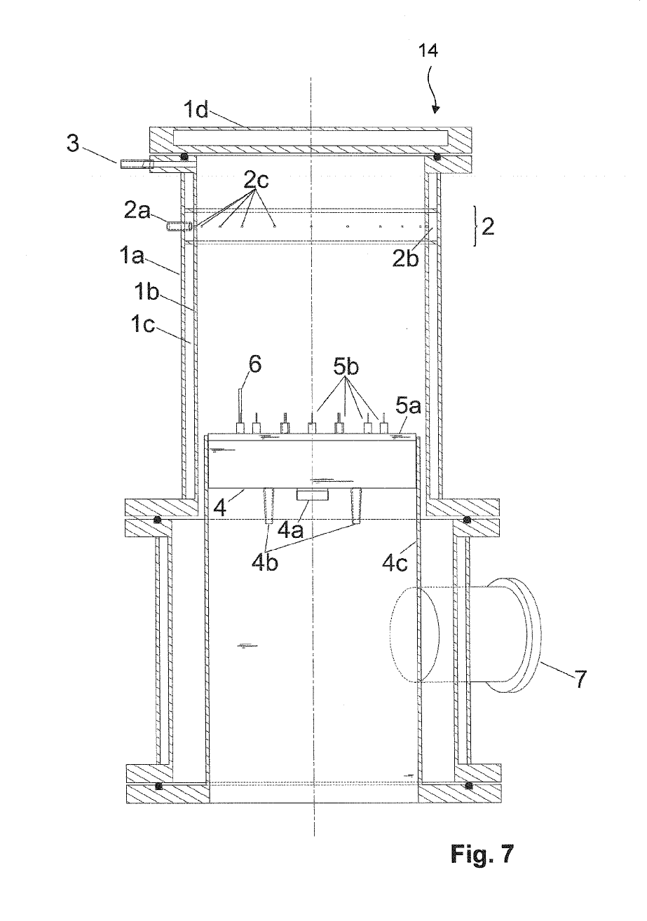

For the coating of stents, a device for the plasma-enhanced chemical vapor deposition was used. A device according to the invention for the plasma-enhanced chemical vapor deposition (PECVD-reactor) is shown in FIG. 7. In the present preferred exemplary embodiment, the process chamber which can be evacuated consists of essentially cylindrical vacuum flange parts with a double wall of chemically resistant- and stainless steel. This wall is formed by an outer wall 1a and an inner wall 1b, between which a ring-like cavity 1c is located. Into this cavity, a fluid heating agent (deionized water) is fed, in order to adjust the temperature of the inner wall lb limiting the treatment zone (T_{Reactor}=50° C.).

The entire cavity is provided with non-depicted guiding means for the heating agent, in order to suitably guide the heating means and thus achieve a homogenous temperature distribution over the inner wall 1b. This is also valid for the double-walled closing lid 1d, the temperature of which can be adjusted, the closing lid enabling the insertion and removal of the stent.

The ring shower 2 for the carrier gas flow with the precursor HMDSO is mounted in the upper region of the cavity 1c. Into this, the vaporous precursor is guided from the precursor reservoir (reservoir temperature T_{H}=36.4° C.) by means of a vacuum stable feed line (feeding temperature T_{L}=45° C.), of which the temperature can be adjusted, via the connecting hub 2a into the ring shower volume 2b. By a suitable selection of the diameter (e.g. 0.2 mm) of the holes 2c in the inner wall 1b, the precursor vapor can homogenously spread in the shower cavity before reaching the treatment zone evacuated to p=14 Pa through the holes. The precursor flow during the coating process is 4 sccm.

The holes 2c are located about 40 mm lower in the present exemplary embodiment than the inlet 3 for the process gas flow. The process gas flow in this example consists of 60 sccm O₂ during the coating process, and of 100 sccm O₂ during the cleaning process.

For the purpose of the coating, up to 18 stents 6 are positioned on the electrically isolating holding elements 5b on the holding device, the stent holding plate 5a. The chemically resistant- and stainless steel plate lies on the cylindrically formed counter electrode, which has a diameter of 145 mm. This electrode 4 is connected in an electrically isolating and vacuum-tight manner with the protecting shield 4c and is held by this in its position in the process chamber, i.e. in the present case about 150 mm beneath the holes 2c. At 20° C., cooling agent (e.g. deionized water) is introduced into the electrode via the inlet- and outlet-ports 4b, and the electrode 4 is supplied with the RF-high voltage (f=13.56 MHz) via a conventional coaxial high-performance-RF-connection 4a (e.g. Huber+Suhner, 7/16).

The process chamber is evacuated by connecting a suitable, typically multi-step vacuum pump to the intake socket 7.

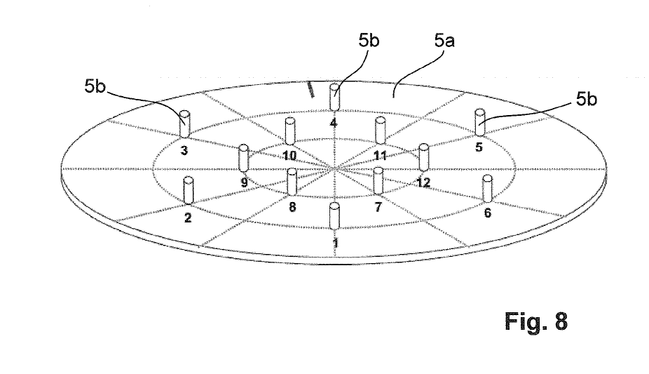

The device used here consists in its core of a cylindrical vacuum chamber, the reactor with a volume of about 8.3 , wherein the portion of the so-called “stent chamber” only makes up about 3 l). The carrier gas (O₂) of the layer-forming agent (HMDSO) needed, among others, for the reaction, is introduced at the head (the upper end) of the device, and flows, at the selected reactor pressure of 0.14 mbar in a laminar manner toward the counter electrode mounted in the lower part of the stent chamber with the stent holding plate (see FIG. 8). The counter electrode with the stent holding plate is provided with an electric supply for the operation of a radio frequency (RF)-discharge.

Therefore, in the RF-mode, the discharge has a direct impact on the deposition process, wherein especially the so-called self-bias of the substrate holder 9 has a superior meaning.

This developing gradient of direct voltage from the plasma to the substrate holder 9 results in high-energy ions from the gas phase striking the growing layer, whereby especially its surface structure can be strongly influenced. The depicted supports to be coated were pre-cleaned before the coating step, wherein the pre-cleaning is advantageous, but not mandatory. The total volume flow during the cleaning was set to 100 sccm. In the present cases a gas volume flow (flow rate) of 100 sccm for oxygen was used (standard volume flow in standard cubic centimeters per minute (sccm)), at a plasma power of 200 W and a cleaning time of 2×10 sec. For the purpose of cleaning, the use of other gas-types, such as for example argon (Ar), ammonia gas (NH₃), hydrogen (H₂) or ethin (C₂H₂) is also possible.

For holding the stents, a stainless, non-magnetic stent holding plate 5a (e.g. a steel plate) can be used, which is provided with holding elements 5b (e.g. pins) (see FIG. 8). In the present case the steel plate 5a has a diameter of 140 mm, wherein for the purpose of simultaneous coating of several stents 6, twelve 5 mm high pins 5b 11 (preferably metal pins) of 1.5 mm diameter are mounted on the steel plate 5a.

The HMDSO used (Sigma-Aldrich, CAS N° 107-46-0) has a boiling point of 101° C., a melting point of −59° C. at a density of 0.764 g/ml at 20° C. The gaseous oxygen used (PanGas AG, O₂ 5.0) has a degree of purity of 99.99999%. As a heat transfer medium (heat exchange agent), deionized water was used.

LIST OF REFERENCE SIGNS

| 1a | outer wall in 14 |
|---|---|
| 1b | inner wall in 14 |
| 1c | cavity in 14 |
| 1d | closing lid in 14 |
| 2 | ring shower |
| 2a | connecting hub |
| 2b | ring shower volume of 2 |
| 2c | hole in 1 or 2 |
| 3 | inlet port |
| 4 | electrode |
| 4a | high-performance-RF- |
| | connection |
| 4b | inlet/outlet |
| 4c | protective shield |
| 5a | stent holding plate |
| 5b | holding element |
| 6 | stent, support |
| 7 | intake socket |
| 8 | support ring of 6 |
| 9 | connecting point of 6 |
| 10 | surface of 6 |
| 11 | sharp edges of 6 |
| 12 | SiOx-coating of 6 |
| 13 | damage in 12 |
| 14 | reactor for coating |

1: A coating for a medical implant, particularly for a vascular stent, comprising silicon dioxide, wherein the thickness of the coating is 40 to 150 nm, and wherein O₂ and hexamethyldisiloxane (HMDSO) are used as reactants for a plasma polymerisation for the production of the coating, characterized in that the HMDSO is incompletely oxidized.

2: The coating according to claim 1, wherein the thickness of the coating is 60-120 nm, preferably 80-100 nm, more preferably in the range of 80 nm.

3: The coating according to claim 1, wherein the coating has a maximal mean defect size of 0.5-2 μm, preferably in the range of 1 μm.

4: A method for the production of a coating according to claim 1, wherein a ratio of [O₂] to [HMDSO] in the range of 10:1 to 40:1, preferably in the range of 10:1 to 20:1, more preferably in the range of 14:1 to 18:1, most preferably in the range of 15:1 is used.

5: The method according to claim 4, wherein 80-95% of the HMDSO is oxidized.

6: The method according to claim 4, wherein a flow rate of O₂ of 120-170 sccm is used, at a flow rate of HMDSO of 5-15 sccm, preferably at a plasma power of 100-300 W, a preferred coating time of 2×4−8 sec and a preferred reactor pressure of 0.1-0.4 mbar.

7: The method according to claim 6, wherein a flow rate of O₂ in the range of 150 sccm is used, at a flow rate of HMDSO in the range of 10 sccm, at a plasma power in the range of 200 W, a coating time in the range of 2×6 sec, and a reactor pressure in the range of 0.2 mbar.

8: A medical implant, particularly vascular stent, comprising a support forming a basic structure and a coating according to claim 1 applied to at least parts of the support and/or produced by a method according to claim 4.

9: The medical implant according to claim 8, wherein the support is synthesized of a material which is difficult to degrade, particularly carbon, PTFE, Dacron, metal alloys, or comprising or consisting of PHA.

10: The medical implant according to claim 9, wherein the support is formed of at least one iron alloy, particularly of stainless steel.

11: The medical implant according to claim 9, wherein the support is formed of a metal having shape memory, particularly of at least one nickel-titanium alloy.

12: The medical implant according to claim 8, wherein the support comprises on its surface a maximum mean defect size of 0.5-2 μm, preferably of in the range of 1 μm.

13: The medical implant according to claim 8, wherein the support has a mean surface roughness Rₐ of at the most in the range of 30 nm, preferably of at the most in the range of 20 nm.

14: A method for the production of a coated medical implant, particularly of a medical implant according to claim 8, comprising the following steps:
providing a support forming a basic structure;
electropolishing the support;
applying a coating comprising silicon dioxide, particularly a coating according to claim 1, by means of a plasma coating process.

15: The method according to claim 14, wherein
as a support a tubular metal blank of stainless steel is provided, which is cut in a laser cutting process and subsequently preferably etched with a solution of deionized water, nitric acid, and hydrofluoric acid; and wherein
the electropolishing of the support is carried out in an electrolyte bath, at a temperature of 70-74 degrees Celsius, a rotational velocity of 2-6 mm/sec, a maximum voltage of 3-4 V, preferably of in the range of 3.5 V, at an electric current of at the most 3-7 A, preferably in the range of 5 A, wherein the duration of the electropolishing is 300-500 sec.

16: The method according to claim 15, characterized by one or more of the following parameters:
that the electrolyte bath contains phosphoric acid, sulphuric acid and distilled water;
that the electropolishing is carried out at a temperature of 70.3-73.5 degrees Celsius;
that the rotational velocity is in the range of 4 mm/sec;
that a voltage of at the most in the range of 3.11 V is applied;
that the duration of the electropolishing is 440-470 sec., preferably in the range of 455 sec.

17: The method according to claim 15, wherein the laser cutting process comprises one or more of the following parameters:
continuous wave pulse transmission;
mean power of 5-9 W, at a power of at the most 80-100 W;
frequency of 5000-8000 revolutions/sec;
shutter speed of 10-12 μs;
energy of 0.8-1.2 mJ;
cutting speed of 2-4 mm/sec;
positioning time of 5-10 mm/sec.
